(19) United States
(12) Plant Patent Application Publication
(10) Pub. No.: US 2009/0038038 P1
(43) Pub. Date: Feb. 5, 2009

(54) APPLE TREE NAMED 'ALVINA'

(75) Inventor: Glynn Edward Fankhauser, Victoria (AU)

Correspondence Address: KLARQUIST SPARKMAN, LLP, 121 SW SALMON STREET, SUITE 1600, PORTLAND, OR, 97204, US

(73) Assignee: Elizabeth Fankhauser

(21) Appl. No.: 11/879,420

(22) Filed: Jul. 16, 2007

(51) Int. Cl. A01H 5/00

(52) U.S. Cl. PLT/162

(57) ABSTRACT

A new apple variety distinguished by very high level of fruit over color, with solid flush and stripes and maturing earlier than comparator Gala selections.

### CROSS REFERENCE TO RELATED APPLICATIONS

Australian PBR application 2006/043 accepted 29^{th} of April 2006

### LATIN NAME OF THE GENUS AND SPECIES OF THE PLANT CLAIMED

Malus Domestica

### VARIETY DENOMINATION

Alvina

### BACKGROUND OF THE INVENTION

The present invention relates to a new and distinct variety of apple tree named ‘Alvina’. The new tree resulted from the discovery of a whole tree mutation in a Gala orchard in 2000. The resulting tree was selected when growing in a cultivated area in Drouin, Victoria, Australia.

### BRIEF SUMMARY OF THE INVENTION

The ‘Alvina’ variety is distinguished from other apple varieties due to the following unique combination of characteristics: very high level of fruit over color, with solid flush and stripes and maturing earlier than comparator Gala selections.

Asexual reproduction of this new variety was performed by budding and grafting and showed that the foregoing characteristics come true to type.

The following detailed description concerns the original tree, ‘Alvina’. The original tree and progeny have been observed growing in a cultivated area in Drouin, Victoria, Australia. Certain characteristics of this variety, such as growth and color, may change with changing environmental conditions (e.g., light, temperature, moisture, nutrient availability, or other factors). Color descriptions and other terminology are used in accordance with their ordinary dictionary descriptions, unless the context clearly indicates otherwise. Color designations are made with reference to The Royal Horticultural Society (R.H.S.) Colour Chart.

### BRIEF DESCRIPTION OF THE DRAWINGS

FIG. 1 is a photograph of representative apples from the ‘Alvina’ variety taken in February 2007 in Droulin, Victoria, Australia.

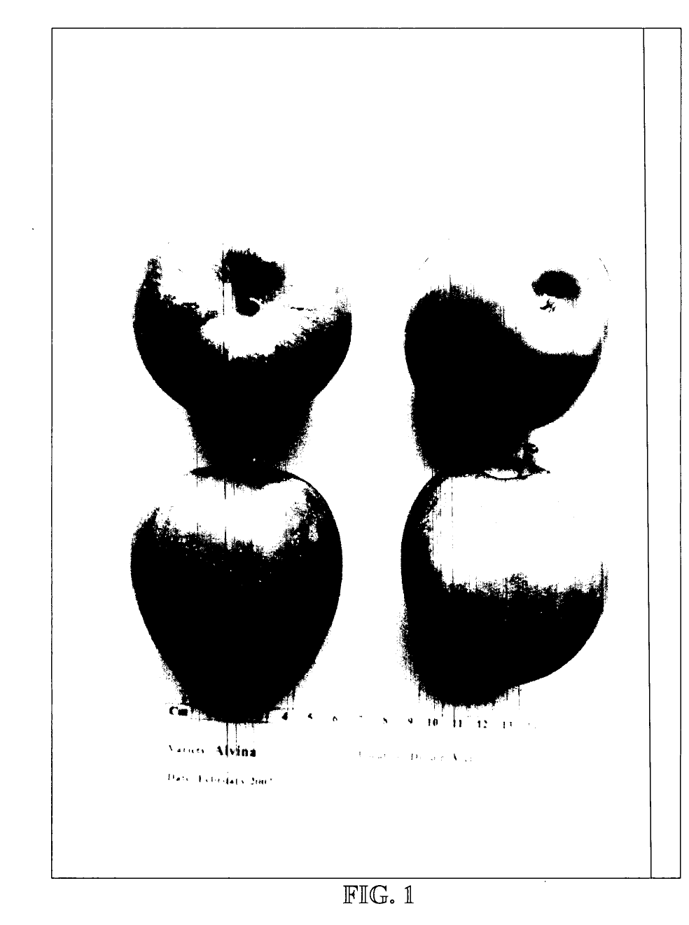

FIG. 2 is a photograph of a cross-sections of representative apples from the ‘Alvina’ variety taken in February 2007 in Droulin, Victoria, Australia.

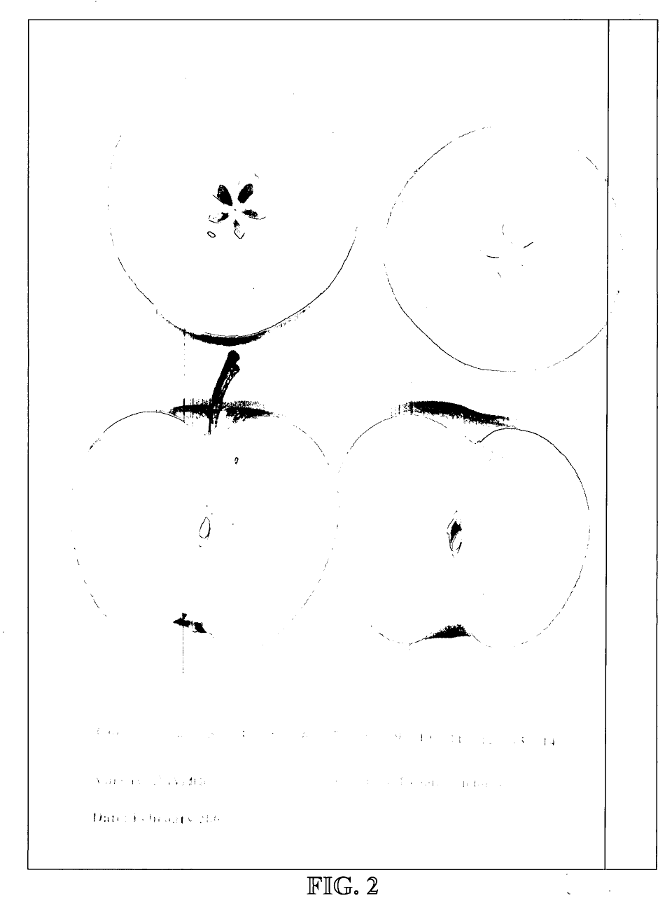

FIG. 3 is a photograph of an ‘Alvina’ tree bearing fruit growing in Droulin, Victoria, Australia.

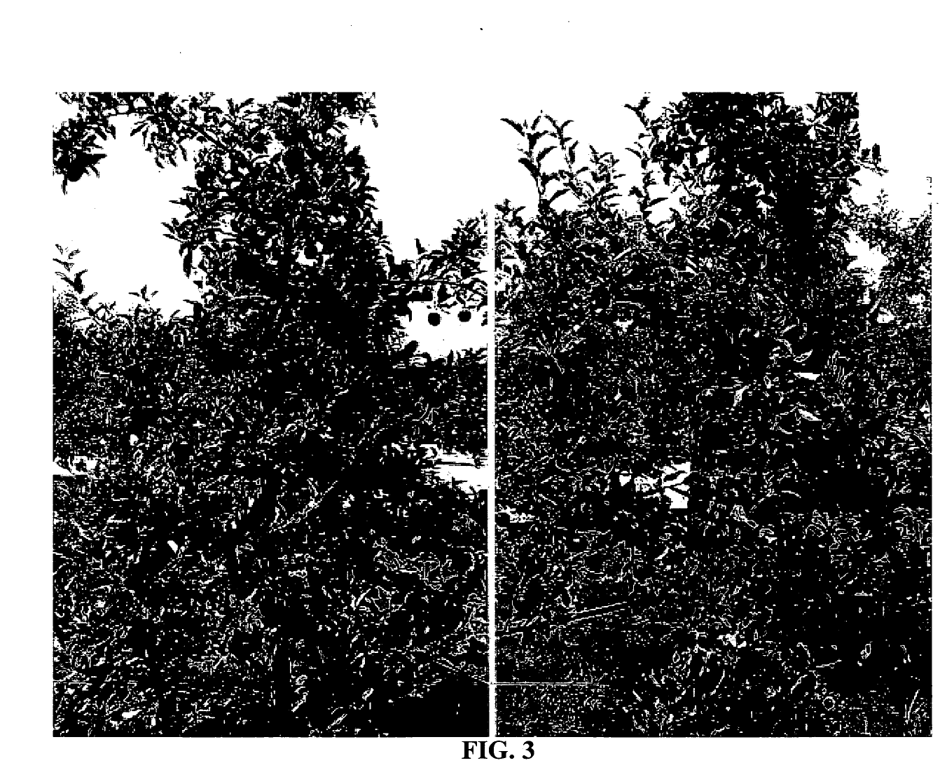

The colors of an illustration of this type may vary with lighting and other conditions under which conditions and, therefore, color characteristics of this new variety should be determined with reference to the observations described herein, rather than from these illustrations alone.

### DETAILED DESCRIPTION

The following detailed description of the ‘Alvina’ variety is based on observations of 10 trees. The observed progeny are trees which were 5 years of age and growing on M26 rootstock in the orchard in Drouin, Victoria, Australia.
Scientific name: Malus Domestica
Tree:
   Vigor.—Medium.
   Overall shape.—Upright spreading.
   Height.—About 8 ft.
   Width.—Overall spread of about 4 ft.
   Branch color.—One-year old branches are Purple (RHS N77) in color.
   Branch pubescence.—Slight.
   Branch lenticels.—medium density, approximately 60 per square inch; on a one year old branch, typical examples of which measured about 0.04 inches in diameter; White (RHS N155A) in color.
   Internodes.—Average internode length is about 1.0 inch on a one-year old shoot.
   Bearing.—Annual.
   Hardiness.—Zones, Australian and other Zones where Gala is adapted.
   Disease resistance.—Alvina has no identified pest and disease resistances.
Leaves: (measurements were obtained from observations of ten typical leaves in autumn)
   Texture.—Leathery.
   Sheen.—Glossy.
   Length.—About 4.0 inches to about 3.0 inches, averaging about 3.7 inches.
   Width.—About 1.8 inches to about 2.3 inches, averaging about 2.1 inches.
   Thickness.—About 0.012 inch to about 0.021 inch, averaging about 0.017 inches.
   Petiole.—About 1.25 inches long; Green-Red in color; about 0.08 inches in Diameter.
   Margin.—Serrate.
   Tip shape.—Acute.
   Stipules.—2 stipules in opposite arrangement; (RHS 144A) in color; Elliptical and narrow shape; length about 0.55 inch long and about 0.1 inch wide.
   Leaf color.—Upper leaf surface: Yellow-Green (RHS 147A). Lower leaf surface: Yellow-Green (RHS 147C). Vein: Greyed-Purple (RHS 185B).
   Pubescence.—Light.
Fruit: (Observations from a limited number of typical fruit in the laboratory)
   Size.—medium, about 3.1 inches long and 3 inches wide.
   Form.—Conical globose.
   Cavity.—1.1 inches wide and 0.5 inches deep.
   Basin.—About 0.4 inch deep and about 1.1 inch wide; pubescence observed weak or absent.
   Stem.—Medium thickness; typical average observed in 10 fruit, about 0.75 inch long and 0.085 inch in diameter; Greyed Purple (RHS 183A) in color.
   Locules.—Open locules.
   Skin.—Intensity of colour is bright.
   Lenticels.—Medium in number.
   Color.—General color effect: Solid Red Flush with Stripes, red in colour over 95% of the fruit Ground color: Greyed Red (RHS 180B) Overcolor: Red (RHS 46A) Russetting: Cheeks, absent, Cavity present, Eye absent.
   Core.—Median bundle area shape; about 1.55 inches long and about 1.25 inches wide.
   Seed.—About 1.3 seeds per cell; Acute shaped; about 0.43 inch long and about 0.24 inch wide; Purple (RHS N77A) in color.

Harvest dates.—Comparative data shown in Table 1.

TABLE 1
Harvest dates for ‘Alvina’ and other Gala varieties in Drouin,
Victoria

| Variety | 2006 harvest dates | 2007 harvest dates |
|---|---|---|
| ‘Galaxy’ Gala | February 9 | February 22 |
| (U.S. Plant Pat. No. 6,955) | | |
| ‘Royal’ Gala | February 22 | February 26 |
| (U.S. Plant Pat. No. 4,121) | | |
| ‘Alvina’ Gala | February 7 | February 19 |

Usage: Fresh consumption

I claim:

1. A new and distinct variety of apple tree, substantially as herein shown and described.
